(19) United States
(12) Patent Application Publication
(10) Pub. No.: US 2012/0303047 A1
(43) Pub. Date: Nov. 29, 2012

(54) VASCULAR CLAMP

(76) Inventor: XUFENG WEI, Catonsville, MD (US)

(21) Appl. No.: 13/118,387

(22) Filed: May 28, 2011

(51) Int. Cl. A61B 17/30

(52) U.S. Cl. 606/158

(57) ABSTRACT

A vascular clamp is provided. The vascular clamp includes a first clamp side and a second clamp side. The first clamp side has a first handle, a first coupling section, a first curved section, and a first jaw section. The second clamp side has a second handle, a second coupling section, a second curved section, and a second jaw section. Further, the first jaw section is structurally coupled to the first curved section; the second jaw section is structurally coupled to the second curved section; and the first clamp side and the second clamp side are coupled together via the first coupling section and the second coupling section such that the first jaw section and the second jaw section form a jaw for partially occluding a posterior or lateral portion of a vessel, and the first curved section and the second curved section form an opening above the jaw for keeping a blood flow of the vessel uninterrupted while the vessel is clamped by the jaw.

### FIELD OF THE INVENTION

This invention generally relates to a vascular clamp and, more particularly, to a vascular clamp for partially occluding a blood vessel.

### BACKGROUND

Accidental bleeding remains a critical issue in a contemporary surgery, especially in a cardiovascular surgery. Bleeding from the posterior or lateral wall of a large vessel during a surgery can be very serious and dangerous, and it can be difficult even for senior surgeons to deal with such bleeding. Unfortunately, such accidents occasionally happen during surgery despite the advances in surgical techniques. For example, such bleeding could be caused by an accidental injury to the posterior or lateral wall or branches of descending aorta being dissected or mobilized; by an unexpected tear of the patent ductus arterials (PDA) being dissected or ligated; or by an unsuccessful end-to-end anastomosis during an operation on a large vessel or a stentless bioprosthetic valve replacement.

When such bleeding does happen, exposing the bleeding site can be difficult because of its deep position and the blood coming from the vessel at a high speed. How to repair the vessel depends on the location of the bleeding and the speed of blood loss. For instance, for minor bleeding from the lateral wall of the vessel, a surgeon could press a finger on the bleeding site and repair it with one or two sutures without too much blood loss. However, for severe bleeding from the lateral wall of the vessel, although the surgeon could repair it by pressing the bleeding site with a finger, a lot of blood loss will usually occur. Moreover, if the bleeding is from the posterior wall of the vessel, even if the bleeding is not severe, it may be hard to repair the vessel without adopting some complicated procedures, such as a cardiopulmonary bypass or circulation arrest under deep hypothermia. Sometimes, the surgeon has to use regular vascular clamps, such as the Debakey vascular clamp, to completely occlude the vessel. However, as this stops the bleeding, it also stops the blood supply to distal organs. If the bleeding is from an unsuccessful end-to-end anastomosis of vessels or grafts, it may be necessary for the surgeon to perform re-anastomosis or to adopt other complicated methods, which may increase the morbidity and mortality of the surgery.

Current vascular clamps for surgeons to perform anastomoses or stop bleeding can be divided into two categories according to the jaws of the clamps: the vascular clamps with a straight jaw and the vascular clamps with a curved jaw. The vascular clamps with a straight jaw, such as peripheral vessel clamps, coarctation forceps, renal artery clamps, and patent ductus clamps, etc., are used for clamping the vessel totally. On the other hand, the vascular clamps with a curved jaw, such as Kay aorta clamps, Beck clamps, Satinsky vascular clamps for vena cava, and Lambert-Kay clamps, etc., can partially occlude the anterior part of the vessel while leaving the posterior part unaffected.

Thus, there is a need for a special vascular clamp that could partially occlude the posterior or lateral part of a vessel while leaving the anterior part of the vessel open. The disclosed methods and systems are directed to solve one or more problems set forth above and other problems.

### BRIEF SUMMARY OF THE DISCLOSURE

One aspect of the present disclosure provides a vascular clamp. The vascular clamp includes a first clamp side and a second clamp side. The first clamp side has a first handle, a first coupling section, a first curved section, and a first jaw section. The second clamp side has a second handle, a second coupling section, a second curved section, and a second jaw section. Further, the first jaw section is structurally coupled to the first curved section; the second jaw section is structurally coupled to the second curved section; and the first clamp side and the second clamp side are coupled together via the first coupling section and the second coupling section such that the first jaw section and the second jaw section form a jaw for partially clamping a posterior or lateral wall of a vessel, and the first curved section and the second curved section form an opening above the jaw for keeping a blood flow of the vessel uninterrupted while the vessel is clamped by the jaw.

Other aspects of the present disclosure can be understood by those skilled in the art in light of the description, the claims, and the drawings of the present disclosure.

### BRIEF DESCRIPTION OF THE DRAWINGS

FIG. 1 illustrates an exemplary vascular clamp consistent with the disclosed embodiments;

FIG. 2 illustrates a top view of an exemplary vascular clamp consistent with the disclosed embodiments.

FIG. 3 illustrates another top view of an exemplary vascular clamp consistent with the disclosed embodiments.

FIGS. 4A-4B illustrate an enlarged view of the tips of two downward blades of an exemplary vascular clamp consistent with the disclosed embodiments;

FIG. 5 illustrates an exemplary use scenario of an exemplary vascular clamp; and

FIGS. 6A-6B illustrate another exemplary use scenario of an exemplary vascular clamp.

### DETAILED DESCRIPTION

Reference will now be made in detail to exemplary embodiments of the invention, which are illustrated in the accompanying drawings. Wherever possible, the same reference numbers will be used throughout the drawings to refer to the same or like parts.

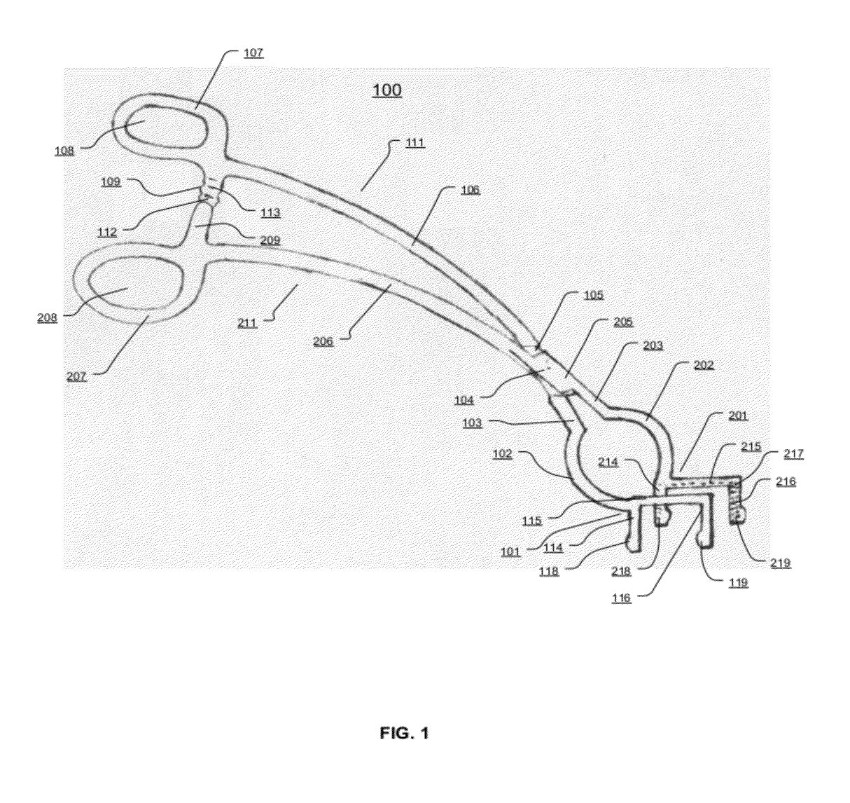

FIG. 1 illustrates an exemplary vascular clamp 100 consistent with the disclosed embodiments. As shown in FIG. 1, vascular clamp 100 may comprise two clamp sides, a first clamp side 111, and a second clamp side 211. The first clamp side 111 and the second clamp side 211 may correspond to each other and may be coupled together to form the vascular clamp 100. Vascular clamp 100 (e.g., the first clamp side 111 and the second clamp side 211, etc.) may be made of any suitable material such as stainless steel or titanium, etc.

Further, the clamp side 111 may also be referred as a left side 111 and may include a handle section 107, a proximal section 106, a coupling section 105, an elongate section 103, a semicircular section 102, and a jaw section 101. Certain sections may be omitted and other sections may be added.

The handle section 107 may include a finger hole 108 to form a finger loop to fit in one or more fingers of a user (e.g., a surgeon), and the proximal section 106 may be a shaft to connect and support between distal end of the handle 107 and the coupling section 105. Further, elongate section 103 extends from the coupling section 105 to the semicircular section 102 which terminates at jaw section 101.

Correspondingly, the clamp side 211 may also be referred as a right side 211 and may include a handle section 207, a proximal section 206, a coupling section 205, an elongate section 203, a semicircular section 202, and a jaw section 201. Certain sections may be omitted and other sections may be added.

The handle section 207 may include a finger hole 208 to form a finger loop to fit one or more fingers of the user, and the proximal section 206 may be a shaft to connect and support between distal end of the handle 207 and the coupling section 205. Further, elongate section 203 extends from the coupling section 205 to the semicircular section 202 which terminates at jaw section 201.

Left side 111 and right side 211 may be coupled together through the coupling sections 105 and 205 using any appropriate fixing mechanism. For example, left side 111 and right side 211 or the coupling sections 105 and 205 may be fixed through a pivot or pin 104 such that left side 111 and right side 211 can rotate around the pin 104. The pin 104 may completely penetrate one or both of left side 111 and right side 211 or may only partially penetrate one or both of left side 111 and right side 211. Further, the coupling sections 105 and 205 may be configured (e.g., surface or direction of the coupling, etc.) such that the left side 111 and right side 211 can move relatively or rotate in a precise and controlled way. For example, the left side 111 and right side 211 can rotate around pin 104 within a same plane.

Being coupled together, left side 111 and right side 211 can rotate into a closed position where the jaw section 101 and the jaw section 201 can contact with each other to form a jaw (i.e., a clamp), while semicircular section 102 and semicircular section 202 may form a circular opening above the jaw. It is understood that the circular shape is used for illustrative purposes, any appropriate shaped opening may be used. For example, any curved shape, such as circular, elliptical, or other shape may be used. Thus, a semicircular section may also be referred as a curved section more generally. When the vascular clamp 100 is applied to a blood vessel, the jaw and the circular opening may be applied to occlude the posterior or lateral portion of the vessel while not interrupting the blood flow in the vessel.

The closed position of the jaw may be maintained and/or controlled by a latch 109 on handle section 107 and a latch 209 on handle section 207. Each latch (i.e., latch 109 and latch 209) may include a latch surface with multiple latching teeth. For example, latch 109 may include a latching surface 112 with multiple latching teeth 113, and latching surface and teeth of latch 209 are not shown but are similarly configured.

The latch 109 is attached on the inner side of handle section 107 and the latch 209 is attached on the inner side of handle section 207 such that the latch 109 of handle section 107 and the latch 209 of handle section 207 are directly opposed when the left side 111 and the right side 211 are pulled together at the handle sections 107 and 207.

When the left side 111 and the right side 211 (e.g., handle sections 107 and 207) are pulled together, the latching surface (e.g., latching surface 112, etc.) of each latch (e.g., latch 109 and latch 209) engages, thereby engaging the latching teeth (e.g., latching teeth 113, etc.) such that the closed position between the left side 111 and the right side 211 (e.g., the jaw section 101 and the jaw section 201, etc.) can be locked.

Further, vascular clamp 100 may have a plurality of closed positions that can be locked so that the user can lock the clamp at a desired closed position. More particularly, any of the multiple teeth on the latch 109 can be interlocked with any of the multiple teeth on the latch 209 such that a combination of lockable positions can be supported. For example, 3 locking teeth on latch 109 may support 3 locking positions. Other interlocking mechanisms may also be used.

The various closed positions may correspond to different pressures can be applied by the jaw of the vascular clamp 100. The closer the closed position, the more pressure the vascular clamp 100 applies. Further, the locked teeth can be released by distorting the handles 107 and 207 (e.g., using the finger loops 108 and 208) to temporarily disengage the locked teeth to allow the vascular clamp 100 to be opened or to be locked at a different position.

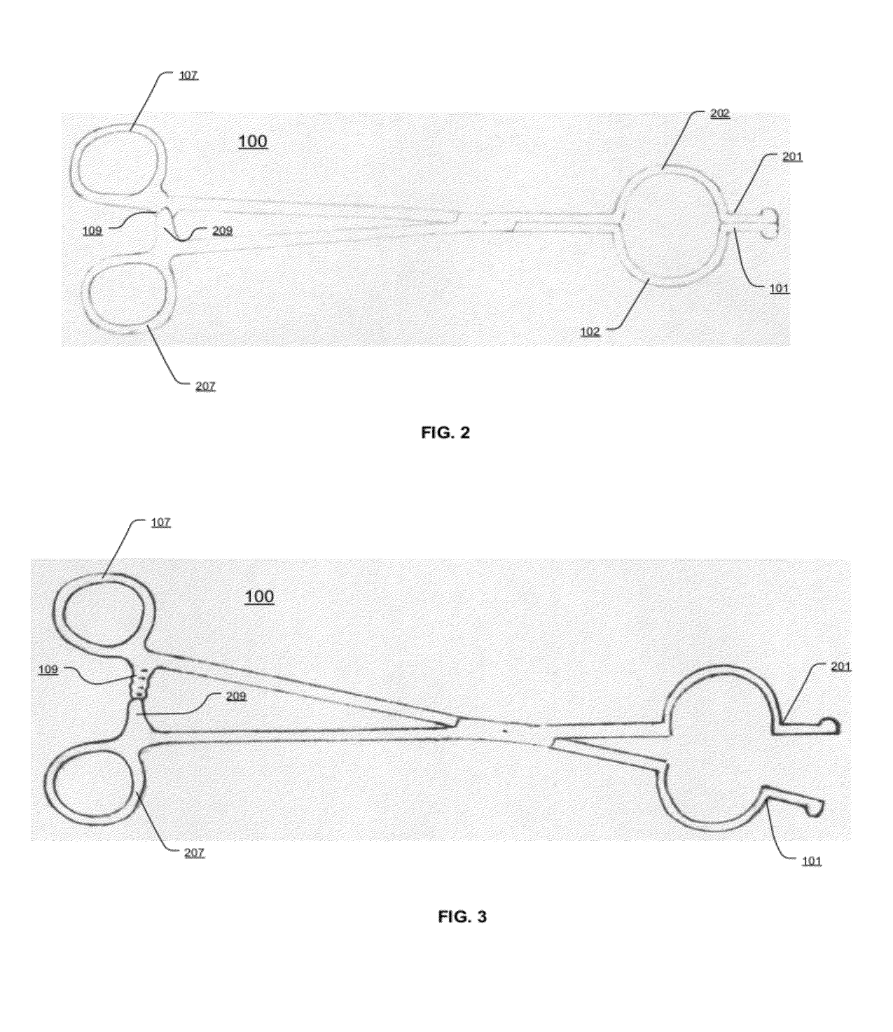

FIG. 2 illustrates an exemplary top view of vascular claim 100 being in a closed position. As shown in FIG. 2, handles 107 and 207 are engaged and the latches 109 and 209 are locked. Further, jaw section 101 and jaw section 201 form a closed jaw and semicircular section 102 and semicircular section 202 form a circular opening above the closed jaw. Different closed positions may also be supported.

FIG. 3 illustrates an exemplary top view of vascular claim 100 being in an open position. As shown in FIG. 3, handles 107 and 207 are disengaged and the latches 109 and 209 are unlocked. Thus, jaw section 101 and jaw section 201 form an open jaw.

Further, each of jaw sections 101 and 201 may have any appropriate structure. Returning to FIG. 1, jaw section 101 may include a horizon blade 115 laterally extended from the semicircular section 102; a first downward blade 114 connecting to the proximal end of the horizon blade 115; and a second downward blade 116 connecting to the distal end of the horizon blade 115. The three blades 114, 115, and 116 of the jaw section 101 form an arch in a plane perpendicular to the plane of the scissor-like motion of the shafts 106 and 206 (or the left side 111 and right side 211). Other shapes or structures may also be used.

Similarly, jaw section 201 may include a horizon blade 215 laterally extended from the semicircular section 202; a first downward blade 214 connecting to the proximal end of the horizon blade 215; and a second downward blade 216 connecting to the distal end of the horizon blade 215. The three blades 214, 215, and 216 of the jaw section 201 form an arch in a plane perpendicular to the plane of the scissors-like motion of the shafts 106 and 206. Other shapes or structures may also be used.

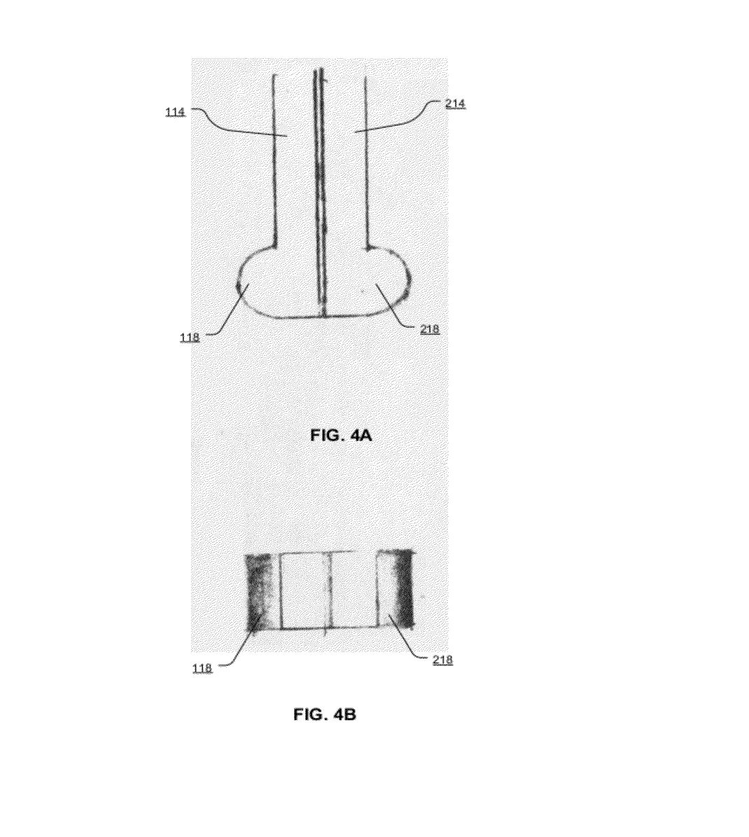

Each downward blade (e.g., downward blade 114, 116, 214, or 216) may have a tip at the end of the blade. For example, downward blade 114 has a tip 118, downward blade 116 has a tip 119, downward blade 214 has a tip 218, and downward blade 216 has a tip 219. FIGS. 4A-4B illustrate exemplary downward blades consistent with the disclosed embodiments. As shown in FIG. 4A, tip 118 of downward blade 114 may be in a round shape or may be a short extension laterally from the end of downward blade 114 such that the end of downward blade 114 or tip 118 can be made blunt. Tip 119 of downward blade 116, tip 218 of downward blade 214, and tip 219 of downward blade 216 may also be similarly made or configured. These ball-like tips increase the contact surface of the blades and make the blades blunt when touching walls of blood vessels. Other shapes may also be used. FIG. 4B shows a bottom view of tip sections of downward blades 114 and 214. The lower end surface of tip 118 may be a combination of a square and a cylindrical surface without any sharp angle.

Returning to FIG. 1, jaw section 101 (e.g., blades 114, 115, and 116) and jaw section 201 (e.g., blades 214, 215, and 216) may each have a contact surface (e.g., contact surface 217). Jaw section 101 and jaw section 201 may form an atraumatic clamping jaw similar to that of a DeBakey or Cooley vascular clamp.

Further, in certain embodiments, the contact surface of jaw section 101 and/or jaw section 201 may be textured to enhance frictional contact. For example, the contact surface may be serrated or ribbed with a plurality of rows of serrated or ribbed teeth aligned along the length of the blades. In certain other embodiments, the contact surface may be made smooth, and the contact surface may be covered with a compliant material, such as expanded silica, rubber, or urethane tubing, etc., to increase frictional contact between the clamping surfaces of jaw sections 101 and 201.

In addition, the blades 114, 115, and 116 of the jaw section 101, and the blades 214, 215, and 216 of the jaw section 201 may be made in various different length and/or angles to match vascular vessels of all types of patients at all ages. The corresponding opening formed by the semicircular section 102 and semicircular section 202 may also be made in different sizes and/or shapes to match the vascular vessels of all types of patients at all ages. The relative position of the opening formed by the semicircular section 102 and semicircular section 202 to the jaw formed by jaw section 101 and jaw section 201 is configured in a way such that a lower portion of a blood vessel can be clamped while an upper part of the blood vessel is not blocked.

Further, structures on the left side 111 and right side 211 may also be configured to make it easy for the user to use the vascular clamp 100 to clamp the lateral wall of a vessel or for other purposes. For example, in certain embodiments, the proximal section (106 or 206) and the elongate section (103 or 203) may be linear. That is, the handle section (107 or 207), the proximal section (106 or 206), the elongate section (103 or 203), and the semicircular section (102 or 202) of a clamp side (111 or 211) may be substantially in the same plane.

In certain other embodiments, the proximal section (106 or 206) and the elongate section (103 or 203) may be non-linear. That is, the handle section (107 or 207), the proximal section (106 or 206), the elongate section (103 or 203), and the semicircular section (102 or 202) of a clamp side (111 or 211) may be in different planes such that a different angle to operate vascular clamp 100 may be achieved. In addition, the elongate sections (103 or 203) may be curved laterally to make it easy to clamp the lateral wall of a vessel or for other purposes.

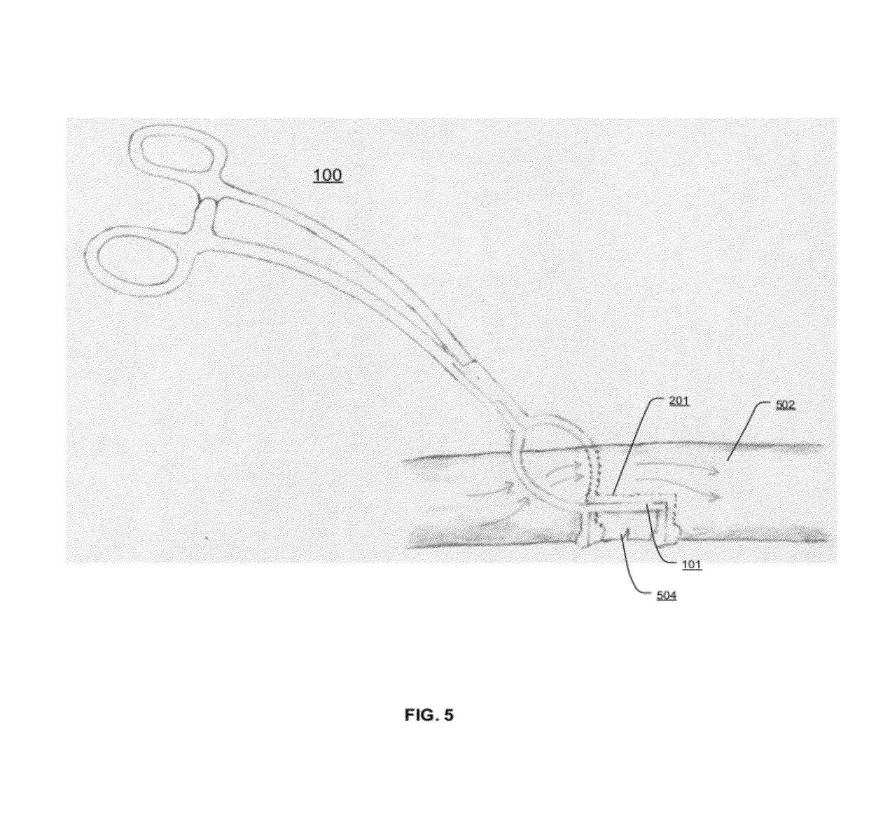

FIG. 5 illustrates an exemplary use scenario of vascular clamp 100 consistent with the disclosed embodiments. As shown in FIG. 5, the vascular clamp 100 is applied on a vessel 502, such as an aorta (ascending, arch or descending), or pulmonary artery. The jaw formed by jaw section 101 and jaw section 201 is clamped to the posterior and lateral walls of the vessel 502 (the posterior wall of the vessel 502 is completed clamped). Thus, bleeding from bleeding part 504 of the vessel 502 can be checked and repaired under a non-bleeding condition. The latch can then be used to select a desired closed position of the clamp.

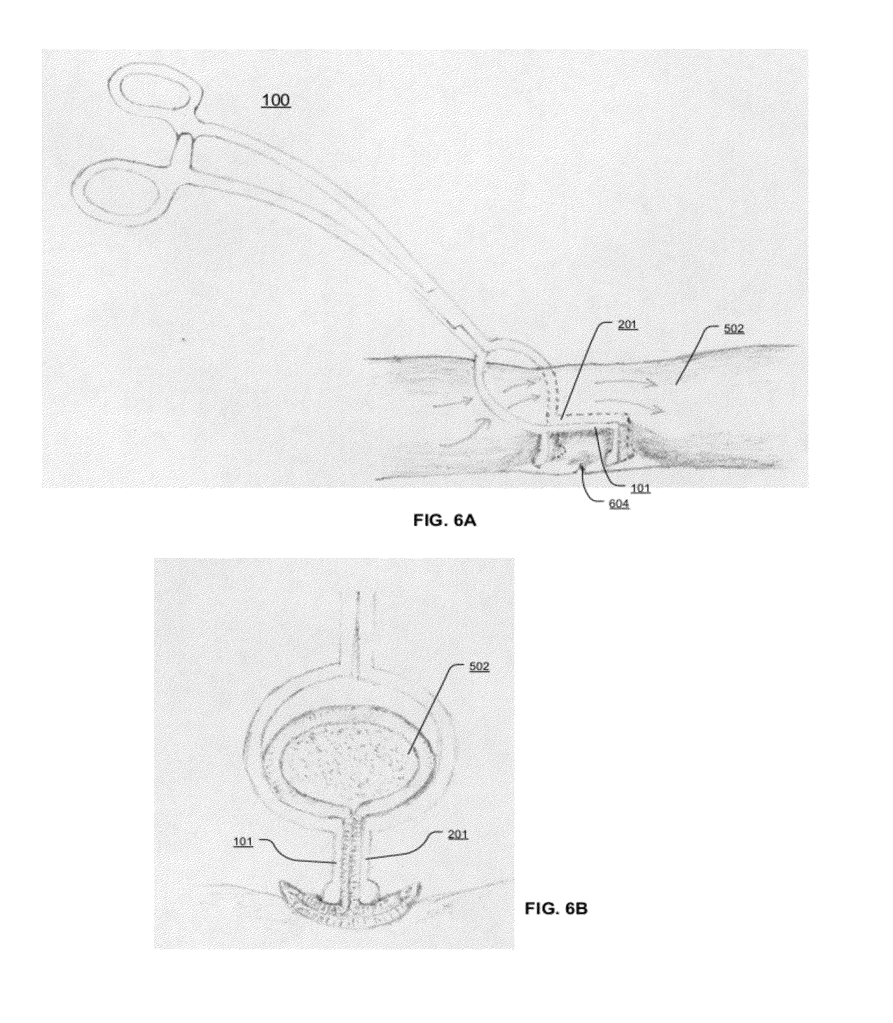

However, if the vessel 502 is adhered to other tissues or organs, such as the posterior wall of the chest for descending aorta, the jaw cannot completely clamp the posterior wall of the vessel 502. FIG. 6A and FIG. 6B illustrate another exemplary use scenario of vascular clamp 100 consistent with the disclosed embodiments.

As shown in FIG. 6A, the vascular clamp 100 is applied on a vessel 502, and the jaw formed by jaw section 101 and jaw section 201 is clamped to the posterior and lateral walls of the vessel 502 (the posterior wall of the vessel 502 is not completed clamped by a properly configured or sized jaw of vascular clamp 100). Thus, the bleeding from vessel 502 can be reduced but not completely stopped. In such situation, the clamp 100 could be gently pressed down until the bleeding stops. If the bleeding point becomes visible, the bleeding point 604 can be repaired directly. If the bleeding point is still invisible, a desired amount of dissection may be performed, and vessel 502 may be re-clamped more deeply until the bleeding point is visible and can be repaired as illustrated in FIG. 5. FIG. 6B shows a cross-section view of FIG. 6A. In all use scenarios, the anterior part of the vessel, i.e., the part above the horizontal jaw, is still open and the blood can pass through and supply the distal organs, because of the opening formed by the semicircular section 102 and semicircular section 202.

By using the disclosed systems and methods, it may be easier to stop bleeding from the posterior or lateral wall of a vessel and to repair the orifice of the bleeding than before. Further, the vessel remains a tube during repair, so the possibility of ischemia and permanent damage to the distal organs may be minimal. In addition, the blood loss may be decreased using the disclosed vascular clamp and corresponding methods, the risk of surgery especially cardiovascular surgery may be reduced, and the stress of the surgeons can be mitigated when accidental bleeding happens. Other advantages and applications may be obvious to those skilled in the art.

What is claimed is:

1. A vascular clamp, comprising:
a first clamp side having a first handle, a first coupling section, a first curved section, and a first jaw section; and
a second clamp side having a second handle, a second coupling section, a second curved section, and a second jaw section;
wherein:
the first jaw section is structurally coupled to the first curved section;
the second jaw section is structurally coupled to the second curved section; and
the first clamp side and the second clamp side are coupled together via the first coupling section and the second coupling section such that the first jaw section and the second jaw section form a jaw for partially occluding a posterior or lateral wall of a vessel, and the first curved section and the second curved section form an opening above the jaw for keeping a blood flow of the vessel uninterrupted while the vessel is clamped by the jaw.

2. The vascular clamp according to claim 1, further including:
a latching mechanism to keep the jaw in a closed position.

3. The vascular clamp according to claim 2, wherein:
the first handle having a first latching surface with a first plurality of latching teeth;
the second handle having a second latching surface with a second plurality of latching teeth; and
the first plurality of latching teeth and the second plurality of latching teeth are configured to latch multiple closed positions of the jaw.

4. The vascular clamp according to claim 1, wherein:
each of the first handle and the second handle includes a finger loop.

5. The vascular clamp according to claim 1, further including:
a first shaft connecting the first handle and the first coupling section; and
a second shaft connecting the second handle and the second coupling section.

6. The vascular clamp according to claim 1, wherein:
the first coupling section and the second coupling section are closely coupled by a pin such that the first clamp side and the second clamp side can rotate around the pin to form the vascular clamp.

7. The vascular clamp according to claim 1, wherein:
the first curved section is semicircular and is connected to the first coupling section via a first elongated section;
the second curved section is semicircular and is connected to the second coupling section via a second elongated section; and
the opening is circular and configured to keep an anterior part of the vessel open.

8. The vascular clamp according to claim 1, wherein each of the first jaw section and the second jaw section includes:
a horizontal blade connected to or laterally extended from respective first or second curved section;
a first downward blade connecting to a proximal end of the horizon blade; and
a second downward blade connecting to a distal end of the horizon blade.

9. The vascular clamp according to claim 8, wherein:
the horizontal blade, the first downward blade, and the second downward blade form an arch in a plane perpendicular to a scissor-like movement plane of the first and second clamp sides.

10. The vascular clamp according to claim 8, wherein:
each of the first jaw section and the second jaw section has a contact surface configured with a plurality of rows of serrated or ribbed teeth aligned along the length of the blades.

11. The vascular clamp according to claim 8, wherein:
each of the first jaw section and the second jaw section has a contact surface covered with a compliant material to increase frictional contact between the first jaw section and the second jaw section.

12. The vascular clamp according to claim 11, wherein:
the compliant material is one of expanded silica tubing, rubber tubing, and urethane tubing.

13. The vascular clamp according to claim 8, wherein:
the first downward blade and the second downward blade each includes a blunt tip.

14. The vascular clamp according to claim 13, wherein:
the blunt tip is in a round shape or a lateral extension.
